(19) United States
(12) Patent Application Publication
(10) Pub. No.: US 2025/0331715 A1
(43) Pub. Date: Oct. 30, 2025

(54) AUTOMATED OCT CAPTURE

(71) Applicant: TOPCON CORPORATION, Tokyo (JP)

(72) Inventors: Tony KO, Cupertino, CA (US); Yuanmu DENG, San Ramon, CA (US); Tzu-Yin WANG, Fremont, CA (US)

(21) Appl. No.: 18/868,475

(22) PCT Filed: May 22, 2023
(86) PCT No.: PCT/US2023/023100

§ 371 Date: Nov. 22, 2024

Related U.S. Application Data
(60) Provisional application No. 63/365,173, filed on May 23, 2022.

(51) Int. Cl. A61B 3/10; A61B 3/00; G06T 7/30; G16H 30/20

(52) U.S. Cl. CPC A61B 3/102; A61B 3/0025; G06T 7/30; G16H 30/20; G06T 2207/10101; G06T 2207/20081; G06T 2207/30041

(57) ABSTRACT

An automated optical coherence tomography (OCT) method that includes receiving an input from a patient, acquiring a reference image of an object indicating a desired scan location and acquiring a real-time image of the object, where the reference image is unique to a patient and remotely acquired. The real-time image is registered to the reference image to determine a desired scan location. An OCT image is automatically acquired at the desiring scan location.

### CROSS-REFERENCE TO RELATED APPLICATIONS

This application claims priority to U.S. Provisional Patent Application No. 63/365,173 filed May 23, 2022 and entitled “AUTOMATED OCT CAPTURE”, the entirety of which is incorporated herein by reference.

### BACKGROUND

Optical coherence tomography (OCT) is a non-invasive imaging technique, often used in ophthalmology. OCT relies on principles of interferometry to image and collect information about an object (such as the eye of a subject). Particularly, light from a source is split into a sample arm where it is reflected by the object being imaged, and reference arm where it is reflected by a reference object such as a mirror. The reflected lights are then combined in a detection arm in a manner that produces an interference pattern that is detected by spectrometer, photodiode(s) or the like. The detected interference signal is processed to reconstruct the object and generate OCT images.

More particularly, structural OCT images and volumes are generated by combining numerous depth profiles (A-lines, e.g. along a Z-depth direction at an X-Y location) into a single cross-sectional image (B-scan, e.g., as an X-Z or Y-Z plane), and combining numerous B-scans into a volume. These depth profiles are generated by scanning along the X and Y directions. En-face images in the X-Y plane may be generated by flattening a volume in all or a portion of the Z-depth direction, and C-scan images may be generated by extracting slices of a volume at a given depth.

One application of OCT imaging is in ophthalmology to diagnose various ocular pathologies and irregularities. During an exam, it is common for a clinician to determine a location that requires additional study and/or imaging. The clinician will typically indicate this location to a technician who performs an OCT scan of the desired location. The generated OCT image is thus dependent on the technician's skill level and the understanding of the clinician's request. That is, if the OCT image is inadequate, e.g., taken at a different location than the desired location, another OCT image would be required.

Additionally, it is common for patients who have eye diseases to be required to have repeated eye scans over a period of time. This is typically accomplished with routine exams/scans at a clinician's office. However, repeated scans can be time consuming and require a clinician's and technician's time to perform.

### BRIEF SUMMARY

According to one example of the present disclosure, a method comprises: receiving an input from a patient, and upon receiving the input: acquiring a pre-existing reference image of an object from a remote database; the pre-existing reference image indicating a desired scan location; acquiring personal information and/or scan settings regarding the patient from the remote database, the pre-existing reference image being unique to the patient and associated with personal information and/or scan settings; acquiring a real-time image of the object; registering the real-time image to the pre-existing reference image; determining the desired scan location on the real-time image based on the registration; and automatically acquiring an OCT image of the object at the desired scan location and according to the acquired personal information and/or scan settings.

In various embodiments of the above example, the pre-existing reference image was originally obtained by a clinician; the real-time image is an OCT en-face image; the method further comprises authorizing the patient based on the input from the patient and acquired personal information; registering the real-time image and determining the desired scan location on the real time image is performed by an machine learning system; the OCT image at the desired scan location is automatically acquired based on whether the desired scan location is within a threshold range of a center of the real-time image; the method further comprises: determining the desired scan location is not within the threshold range of the center of the real-time image, acquiring a second real-time image of the object, registering the second real-time image to the reference image, and determining the desired scan location on the second real-time image based on the registration of the second real-time image; the scan settings comprise a patient-specific scan pattern and the OCT image is automatically acquired according to the scan pattern; the method further comprises aligning the OCT imaging system according to the desired scan location; and/or the object is an eye.

Accordingly to another example, a system comprises an optical coherence tomography (OCT) imaging system; one or more processors collectively configured to: receiving an input from a patient, and upon receiving the input: acquire a pre-existing reference image of an object from a remote databased in response to an input from a patient, the pre-existing reference image indicating a desired scan location; acquire personal information and/or scan settings regarding the patient from the remote database, the pre-existing reference image being unique to the patient and associated with personal information and/or patient-specific scan settings; acquire a real-time image of the object; register the real-time image to the reference image; determine the desired scan location on the real-time image based on the registration; and automatically acquire an OCT image of the object at the desired scan location with the OCT imaging system according to the acquired personal information and/or scan settings.

In various embodiments of the above example, the reference image was originally obtained by a clinician; the real-time image is an OCT en-face image acquired with the OCT imaging system; the one or more processors are further collectively configured to authorize the patient's use of the system based on the input from the patient and the acquired personal information; the real-time image is registered to the reference image by one or more processors configured as an machine learning system; the OCT image at the desired scan location is automatically acquired based on whether the desired scan location is within a threshold range of a center of the real-time image; the one or more processors are further collectively configured to: determine the desired scan location is not within the threshold range of the center of the real-time image, acquire a second real-time image of the object, register the second real-time image to the reference image, and determine the desired scan location on the second real-time image based on the registration of the second real-time image; the scan settings comprise a patient-specific scan pattern, and the OCT image is automatically acquired according to the scan pattern; the one or more processors are further collectively configured to: align the OCT imaging system according to the desired scan location; and/or the object is an eye.

### BRIEF DESCRIPTION OF THE SEVERAL VIEWS OF THE DRAWING

FIG. 1 illustrates an example schematic of an optical coherence tomography system of the present disclosure.

FIGS. 2A and 2B illustrate examples of automated imaging terminals of the present disclosure.

FIG. 3 illustrates an example method of the present disclosure.

FIG. 4 illustrates an example method of the present disclosure.

FIGS. 5A, 5B, and 5C illustrate example reference images.

FIG. 6 illustrates an example registration technique of the present disclosure.

FIG. 7 illustrates an example image depicting a proximity threshold.

FIG. 8 illustrates an example series of OCT images.

FIG. 9 illustrates example real-time images.

### DETAILED DESCRIPTION OF THE DRAWING

Considering the above, the present disclosure relates to automated image capture, particularly OCT image capture. More particularly, the present disclosure relates to automated OCT imaging, for example, by using a reference image.

Using the methods and devices described below, OCT images can be acquired without manual assistance or under the direction of a technician. The use of automated OCT imaging can reduce errors caused by manually choosing the scan location, e.g., imaging the wrong location. Additionally, automated OCT scans can facilitate periodic OCT imaging (e.g., to monitor disease progression, post-surgical analysis, and the like) without the need of a technician/clinician to perform the scan. This saves time for the patient and technician/clinician. Furthermore, automated OCT imaging allows for a more convenient process for the patient, who is not dependent on the operating hours of a clinic or the availability of the technician/clinician.

Generally, automated OCT imaging improves efficiency at least in part because it relies on reference images unique to each patient. This can reduce the scanning time, increase the success rate of generating acceptable images, and reduces the number of necessary scans. These unique reference images are used as ground truths of known pathologies—in other words, as representing known locations of pathologies for the patient. By contrast, for example, a “one size fits all” solution may utilize raster scanning techniques for scanning the entire eye, since individual pathologies about the patient are not necessarily known to the automated imaging system. As a result, such systems and methods have a lower success rate, can require more scans and scan time, and are generally less efficient. Using unique reference images can produce OCT images that are concentrated on the pathology of the specific patient and provide automated custom imaging scans for the patient without a clinician.

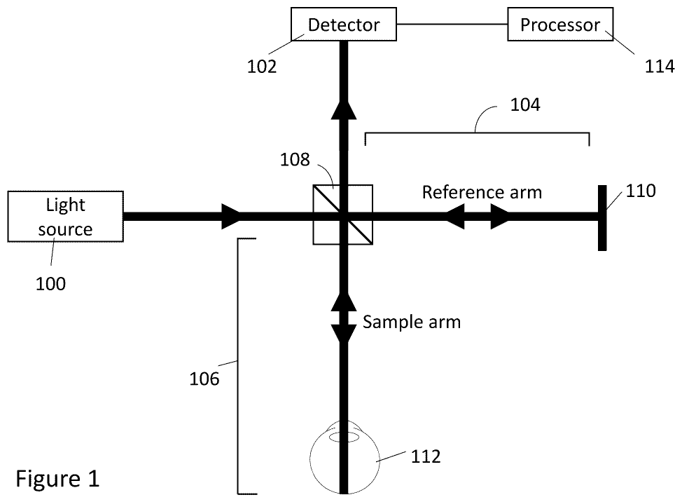

With reference to FIG. 1, an OCT imaging system includes a light source 100. The light generated by the light source 100 is split by, for example, a beam splitter (as part of interferometer optics 108), and sent to a reference arm 104 and a sample arm 106. The light in the sample arm 106 is backscattered or otherwise reflected off an object, such as the retina of an eye 112. The light in the reference arm 104 is backscattered or otherwise reflected by a mirror 110 or like object. Light from the sample arm 106 and the reference arm 104 is recombined at the optics 108 and a corresponding interference signal is detected by a detector 102. The detector 102 can be a spectrometer, photo detector, or any other light detecting device. The detector 102 outputs an electrical signal corresponding to the interference signal to a processor 114, where it may be stored and processed into OCT signal data.

The processor 114 may then further generate corresponding structural or angiographic images or volume, or otherwise perform analysis of the data. The processor 114 may also be associated with an input/output interface (not shown) including a display for outputting processed images, or information related to the analysis of those images. The input/output interface may also include hardware such as buttons, keys, or other controls for receiving user inputs to the system. In some embodiments, the processor 114 may also be used to control the light source and imaging process.

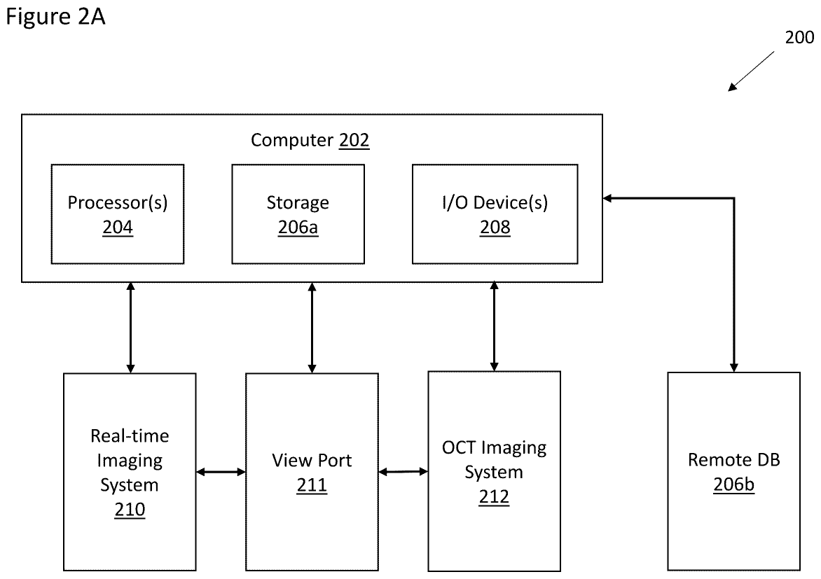

FIG. 2A illustrates an automated imaging terminal 200 of the present disclosure. The imaging terminal 200 shown therein comprises a computer 202 comprising at least one processor 204, local storage 206a, and input/output (I/O) devices 208. The I/O devices 208 can be any input/output devices that allow for communication and selection by a user or patient, for example, a keyboard, a mouse, selection buttons, an LED display, a touchscreen, or the like. In some embodiments, the I/O device 208 comprises a wireless communication device that can communicate using wireless communication standards, such as Bluetooth or Wi-Fi, and communicate to a mobile device, such as a mobile phone. The I/O devices 208 can also communicate to a remote database 206b using a wireless communication standard, ethernet, or via an internet connection. The remote database 206b can be a cloud database, an on-premises database, or the like.

The automated imaging terminal 200 further comprises a dedicated real-time imaging system 210 (such as a Fundus camera, IR camera, SLO camera, or the like) and an OCT imaging system 212. The OCT imaging system 212 can be like the one discussed above and illustrated in FIG. 1. The real-time imaging system 210 and the OCT imaging system 212 can share a view port 211, which can be accessed by a patient to use the automated imaging terminal 200. The view port 211 can comprise at least optics configured to allow the patient's eyes to be imaged by both the real-time imaging system 210 and the OCT imaging system 212.

In some embodiments, such as those shown in FIG. 2B, the automated imaging terminal 200 does not include a dedicated real-time imaging system 210. Instead, the OCT imaging system 212 also serves as a real-time imaging system by acquiring a real-time high-speed en-face OCT image, or the like.

The computer 202 can communicate to and receive information from the real-time imaging system 210 and the OCT imaging system 212. For example, the images acquired by the real-time imaging system 210 and OCT imaging system 212 can be analyzed by the at least one processor 204 (with processing distributed across one or more processors in any manner), stored on the local storage 206a, and/or stored on remote database 206b. The at least one processor 204 can also adjust various camera functions and alignments of the imaging systems 210, 212 with the view port 211 using computer generated commands or controlling physical actuators and motors.

The automated imaging terminal 200 can be in the design of a kiosk, or the like, and be placed in a public location for ease of access. A patient can walk up to a public automated imaging terminal 200 to get regular eye scans at their convenience, e.g., without the need to schedule an appointment with a technician or clinician. The automated imaging terminal 200 can automatically acquire OCT images from desired scan locations indicated in a reference image that is unique to the patient without operator input.

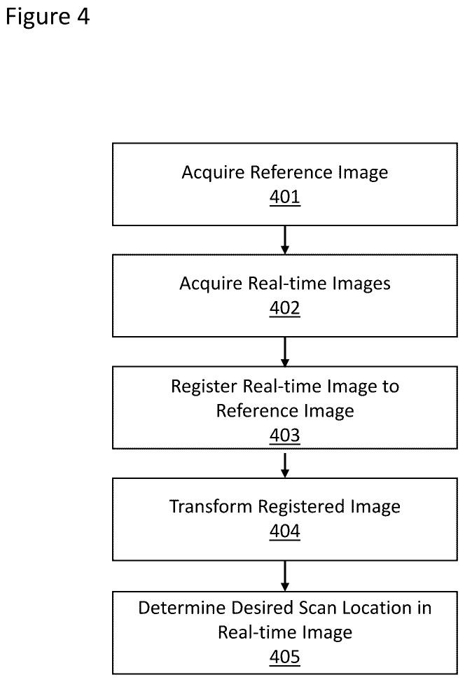

The reference image can be varying types and acquired by various methods. For example, as shown in FIG. 5A, a clinician can manually mark a desired scan location in a chart drawing 510. The chart drawing 510 can be a rudimentary drawing or simplified depiction of a human eye, and marked (e.g., with an ‘X’) to indicate a desired scan location relative to anatomical landmarks. Additionally or alternatively, the reference image can be acquired using a fundus camera that generates a fundus image 512, as shown in FIG. 5B. This fundus image can similarly be marked (e.g., with an ‘X’) to indicate the desired scan location. The reference image can also be an OCT image (e.g., an en-face image, a structural C-scan, an angiographic image, or the like) 514, as shown in FIG. 5C. The en-face OCT image 514 may be acquired using an OCT imaging system such as that illustrated in FIG. 1. As above, the OCT image 514 can be marked (e.g., with an ‘X’) to indicate the desired scan location.

The reference images can represent an a priori knowledge of the pathology of the patient, as described above. In some embodiments though, the reference images may be collected and analyzed by the automated imaging terminal 200 itself or a like system.

In any event, the scan location is marked on the reference image and guides later OCT imaging. In the example of an a priori reference image, the scan location may be marked manually by a clinician or automatically identified by analysis of the reference image. A clinician can manually mark the reference images using computer software or hand marking a copy of the fundus photo using writing utensils. The desired scan locations can be indicated using colored pixels or other marks, could be saved as coordinates on an X-Y axis (e.g., as digital coordinates), saved as specific pixel location information, or the like. Reference images can be patient-specific, and thus unique to each patient. For example, a reference image can indicate a specific desired scan location to observe a particular patient's retinopathy. In some embodiments, the reference images can indicate a generic location, for example, an indication for a desired scan location near the optical nerve to observe the progression of glaucoma.

Scan locations may be automatically determined and marked, for example, based on an analysis of the reference image. In some embodiments, image processing techniques, such as computer vision and/or machine learning, can be used by the automated imaging terminal 200 to determine regions of interest of a real-time fundus image acquired by the real-time imaging system 210 or a real-time en-face OCT image acquired using the OCT imaging system 212. The computer 202 and/or processors 204 can use such image processing techniques including computer vision and machine learning. The region of interest or desired scan location can be a patient's particular pathology.

For example, the reference image may be input to machine learning system trained to identify regions of interest based on abnormalities in the image. Such techniques may be those described in U.S. Pat. No. 11,132,797, titled AUTOMATICALLY IDENTIFYING REGIONS OF INTEREST OF AN OBJECT FROM HORIZONTAL IMAGES USING A MACHINE LEARNING GUIDED IMAGING SYSTEM, the entirety of which is incorporated herein by reference; and/or described in U.S. patent application Ser. No. 16/552,467, titled MULTIVARIATE AND MULTI-RESOLUTION RETINAL IMAGE ANOMALY DETECTION SYSTEM, the entirety of which is incorporated herein by reference.

Acquisition and marking of the reference images may be performed during an examination of a patient, and further saved for later use, for example, within a patient's physical file or virtual file. The virtual file can be stored on a computer's local memory, an on-premise database, or remote database 206b, such as cloud based storage. For instance, a patient can have a reference image on file with their clinician. The clinician can indicate a desired scan location on said reference image using the techniques described above. The reference image with a desired scan location can be stored on a cloud or otherwise remote database 206b for access by the automated imaging terminal 200.

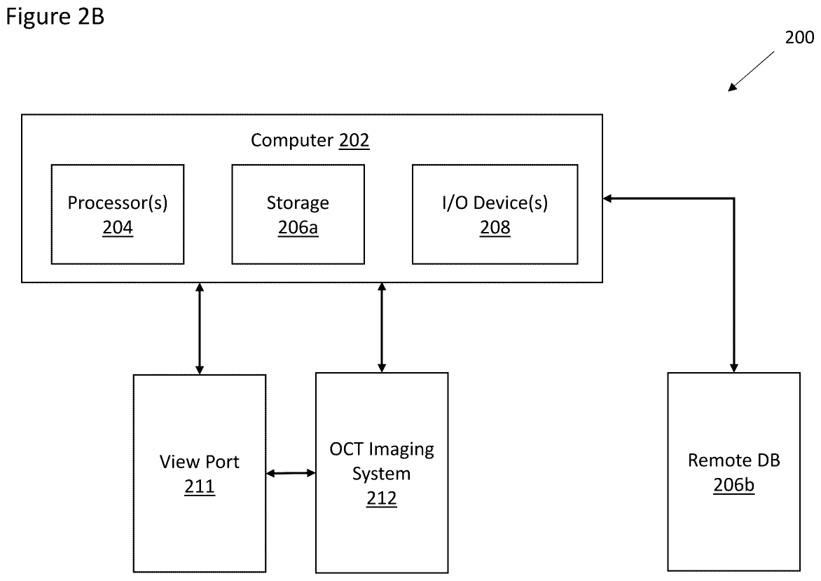

Referencing FIG. 3, the patient can initiate automated capture 301 by using buttons on the device, such as a start button, or by accessing the automated imaging terminal 200 using a personized login. In some embodiments, the patient can use their personal mobile phone to scan a machine-readable optical image, e.g., a QR code, located on or near an automated imaging terminal 200. The QR code can direct the user to a web application or mobile app or generate a text message, where the patient can sign into their personal account and indicate at which automated imaging terminal 200 they are located. For instance, the patient can indicate a location by using a global position system (GPS), a prompt from the application, the GPS location of the automated imaging terminal 200, or a unique QR code that is associated with a particular automated imaging terminal 200.

The automated imaging terminal 200 can then communicate to a remote database 206b via the internet or other network to acquire the patient's reference image, personal information, scan settings, and the like 302. For example, scan settings may be patient-specific and can include resolution, brightness, saturation, contrast, size, scan patterns, or similar image settings that can be used by the real-time imaging system 210 or the OCT imaging system 212 in acquiring images/scans. Personal information can include a name, gender, age, height, weight, medical history and/or pathological information, and the like. Additionally, for example, further instructions can include a starting center location for the real-time imaging system 210 or the OCT imaging system 212, an indication of how many OCT images or scans should be acquired, type of registration method, or other information pertaining to acquiring the real-time images or OCT images.

In some embodiments, the use of the automated imaging terminal 200 may first require authorization of the patient. Authorization can be determined using various methods. For example, the automated imaging terminal may authorize a patient if the patient has a reference image on file with the owner/operator of the automated imaging terminal 200. Particularly, after the patient initiates automated capture 301, the automated imaging terminal 200 can then communicate to a remote database 206b and include patient information, such as a patient ID, login, name, address, or the like. The remote database 206b can use this patient information to determine if the patient is affiliated with the owner/operator of automated imaging terminal 200. For instance, if the automated imaging terminal 200 is owned/operated by a clinician or a service provider for the clinician, the automated imaging terminal 200 would determine whether the patient has personal information or a reference image on file with that clinician or service provider.

This authorization may be facilitated through local storage 206a and/or remote database 206b, which can store the patient information and/or reference images for a clinician or service provider. Thus, the patient information input by a patient at the automated imaging terminal 200 can simply be compared with records stored at the local storage 206a and/or remote database 206b to identify a match. Once a match is determined, the patient may be alerted that they have been authorized and use of the automated imaging terminal 200 may be unlocked and the patient may continue with use of the automated imaging terminal 200 to automatically capture OCT images.

If no corresponding patient information is determined, the patient may be alerted and, for example, asked to contact their clinician (e.g., to schedule an appointment). In some embodiments, a clinician may be suggested or automatically contacted by the automated imaging terminal 200. In some embodiments, the automated imaging terminal 200 may be utilized to collect a reference image for that patient that is then analyzed in real-time (locally or by a remote service) to facilitate further imaging, or transmitted to a clinician for further analysis. The automated imaging terminal 200 may also recommend or automatically suggest such a clinician, and/or may request clinician information from the patient.

Even if a patient information and/or reference image is stored, additional information may be requested to authorize the patient. For example, a clinician can perform regular reviews of their patient files and indicate in the stored patient information the acceptability of the reference image. For instance, if the reference image on file is determined to be old, out of date, of poor quality, associated with a different clinician, or the like, the clinician can indicate this with the patient information. The existence of such information may prevent authorization of the patient's use of the automated imaging terminal 200, and also cause the automated imaging terminal 200 to alert the patient to such issues and automatically contact the clinician or suggest a clinician for the patient to contact.

In some embodiments, the owner/operator of the automated imaging terminal 200 offers access to the automated imaging terminal 200 as a service. For example, the owner/operator can provide this service to a plurality of clinicians, where the automated imaging terminal 200 can service any of those clinician's patients. The automated imaging terminal 200 can acquire additional information 302 that indicates whether the clinician still subscribes to the owner's/operator's service and can authorize patient use based on this information. In some embodiments, each patient is subscribed to the service, which grants them access to the automated imaging terminal 200. Authorization may thus also be based on the patient's subscription.

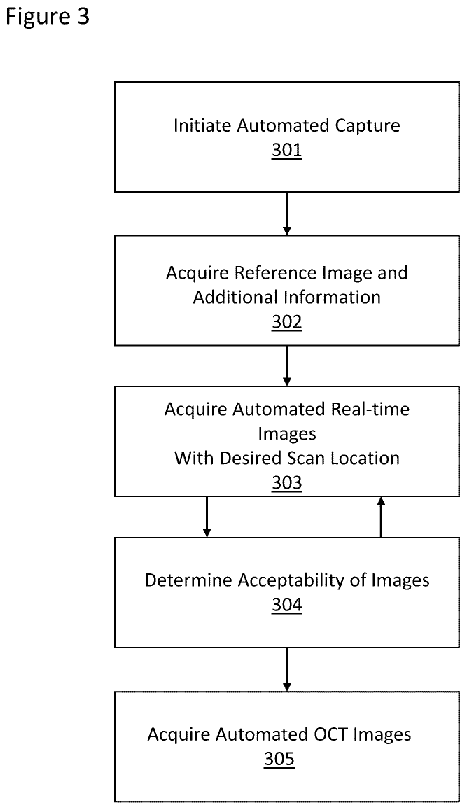

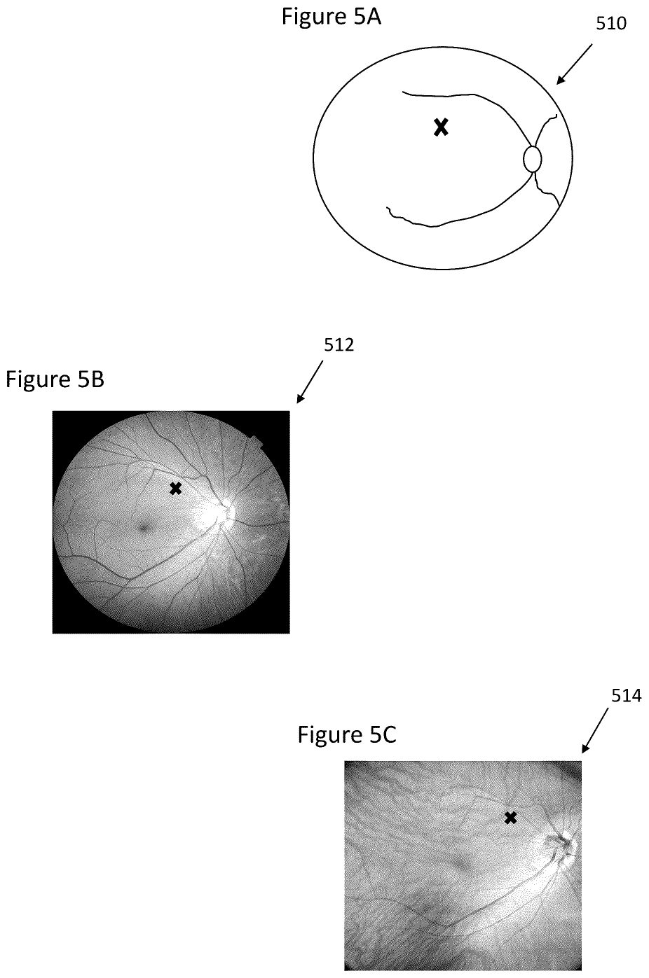

The automated imaging terminal 200 can use the acquired reference image and other information 302 to acquire automated real-time images with a desired scan location 303. For instance, using the example method described in FIG. 4, the automated imaging terminal 200 can acquire reference image 401 by way of a remote database 206b and store them in local storage 206a. The automated imaging terminal 200 can then acquire real-time images 402 using the real-time imaging system 210. With reference to FIGS. 4 and 6, the method can acquire real-time images 402 from the real-time imaging system 210 of the automated imaging terminal 200. For instance, real-time images can be acquired from a fundus camera, infrared video camera, scanning laser ophthalmoscopy images, en-face OCT images, or the like.

To help ensure proper imaging, the automated imaging terminal 200 can use the real-time imaging system 210 and processor(s) 204 to determine whether the patient's eyes is aligned within the view port 211. In some embodiments, using machine learning techniques and the real-time imaging system 210, the processor(s) 204 can detect the macula of each of the patient's eyes to determine if the patient is centered within the view port 211. If the patient is not centered within the view port 211, the processor 204 can use the I/O devices 208 to notify the patient to adjust, via a sound notification, voice command, or visual indication. An LED or like display can be used to give a visual indication of how to center the patient's eyes. For instance, circles displayed on the LED display can represent the desired position of the patient's eyes can be displayed and a second set of circles representing the real-time positioning of the patient's eyes can be present to provide real-time feedback for the patient on how to adjust to the desired positioning. In other embodiments, a ‘bullseye’ can be displayed to guide the patient to align themselves within the view port 211. Other alignment techniques can be used, such as, displaying a real-time video feed, a light indicating the center or focal point of the imaging device, a ring of lights, audible feedback, or the like. The processor 204 can use software that implements image processing techniques and/or machine learning techniques to determine when a patient is correctly aligned.

The acquired real-time images 402 are then adjusted to register 403 the real-time image 603 and reference image 601. Image registration 403 can transform different sets of data into one coordinate system and can allow images taken of a similar location, of the same patient at different times, at different perspectives to be aligned. Image registration can be accomplished by various methods, including feature-based, intensity-based, or the like. In some embodiments, the example method uses a feature-based registration technique to identify the same anatomical structure 602 found in two images corresponding with each other spatially (similar locations). Once the same structure has been identified, the two images may be related by the relative locations of the anatomical structure 602.

As shown in FIG. 6, the example feature-based registration technique uses a reference image 601 with a desired scan location 604 and an anatomical feature 602, and a real-time image 603 comprising a scan center 608 and the anatomical feature 602. The example method can use a feature-based registration technique 610 to relate the anatomical feature 602 of real-time image 603 to the anatomical feature 602 of reference image 601. The anatomical feature 602 can be various parts of the eye, for example, the macula, optic nerve, vascular, or the like.

Feature-based methods can establish a relationship between multiple features or distinct points within images to accomplish geometrical transformations to map the real-time image 603 to the reference image 601. For example, the feature-based registration technique 610 can include various image transformations to orient at least one of the images, such as rotation, scaling, translation and other affine transformations to map or fit the real-time image 603 onto the same coordinate system as the reference image 601. Other transformation methods can also be used, such as, nonrigid transformations, multi-parameter transformations, or the like. Transformations can be accomplished using image processing and/or registration techniques, and/or machine learning techniques. Such techniques may include, for example, feature-based alignment, model fitting alignments, and pixel-based alignments.

Registering the real-time image 603 with the reference image 601 can generate a registered image 605 where the real-time image 603 is mapped to the reference image 601 and can preserve the desired scan location 604 of the reference image 601 and the scan center 608 of the real-time image within the registered image 605. That is, the registered image 605 contains the desired scan location 604 of the reference image 601, in relationship to the scan center 608 of the real-time image 603. In some embodiments the transformations may be made directly to the real-time image, and thus no additional registered image is generated.

The example method can then transform the registered image 404 using transformations 612 to acquire the desired scan location 604 in the real-time image 603. In some embodiments, the inverse of the transformations used in the registration process 610 are used to acquire the desired scan location 604 in a real-time image 603. For example, if during the registration process 610 the real-time image is rotated by 45° clockwise to register the real-time image 603 onto the reference image 601, during the transformation of the registered image 605, transformation 612 can include a rotation by 45° counterclockwise, i.e., the inverse of the transformation during the image registration process. The transformation process 404 preserves the desired scan location 604 from reference image 601, while only including pixel information from real-time image 603. In other words, the registration-transformation process translates the desired scan location onto the real-time image 603.

With the desired scan location associated with the real-time image, the coordinates or location information of that desired scan location (e.g., relative to a center of the view port 211 and resulting images) can then be acquired and/or stored for future use, for instance, in local storage 206a or the remote database 206b.

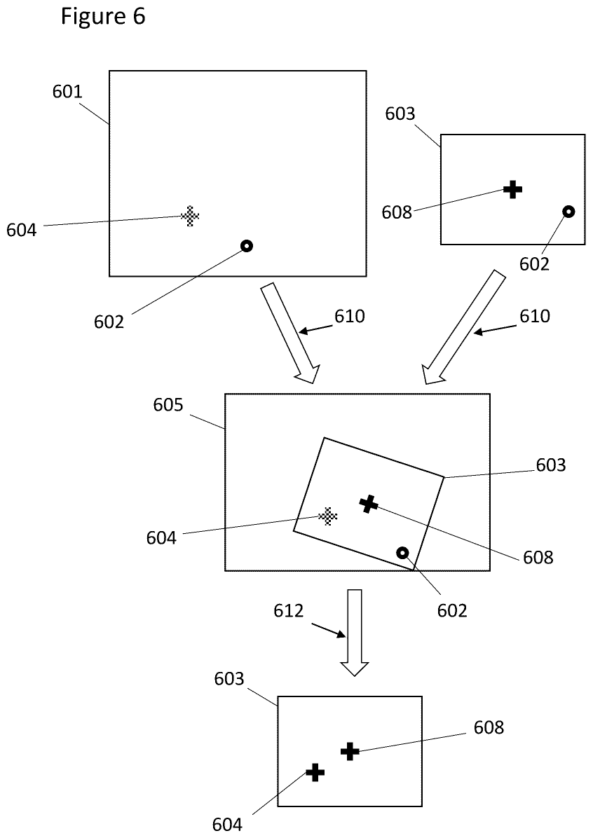

Referring back to FIG. 3, the automated imaging terminal 200 can determine the acceptability of the real-time images based on a number of factors such as the proximity to the desired scan location, the scan instructions, the noise levels, or the like. For instance, depending on the distance from the scan center 608 and the preserved desired location 604, the view port 211 and/or real-time imaging system 210 can be adjusted to relocate the scan center 608 closer to the desired scan location 604. The real-time imaging system 210 can then acquire another real-time image of the new location, and repeat the example method as described in FIG. 4 until the scan center 608 of the real-time image is within an acceptable vicinity to the desired location 604. For example, referencing FIG. 7, a real-time image 700 with a preserved desired location indicator 702 and scan center 706, can be found acceptable because the desired location indicator 702 is within a predetermined proximity threshold 704 (e.g., a number of pixels or determined distance).

The automated imaging terminal 200 can further indicate to the patient whether the real-time image acquired from the real-time imaging system 210 is acceptable by using various indication methods, for example, lights generated on the automated imaging terminal 200, using the I/O devices 208 to generate notifications or voice commands, or similar actions. For instance, if the patient is blinking (or the eye is otherwise unstable) and preventing the real-time imaging system 210 from taking an acceptable image, the I/O devices 208 can use a voice command to instruct the patient to hold their eyes open. The processor 204 can use image processing techniques and/or machine learning techniques for detecting eye movement, instability, and/or blinking. For instance, the processor can use image detection software to detect blinks indicated by a real-time image comprising large dark bands or dark areas within a fundus image or enface image.

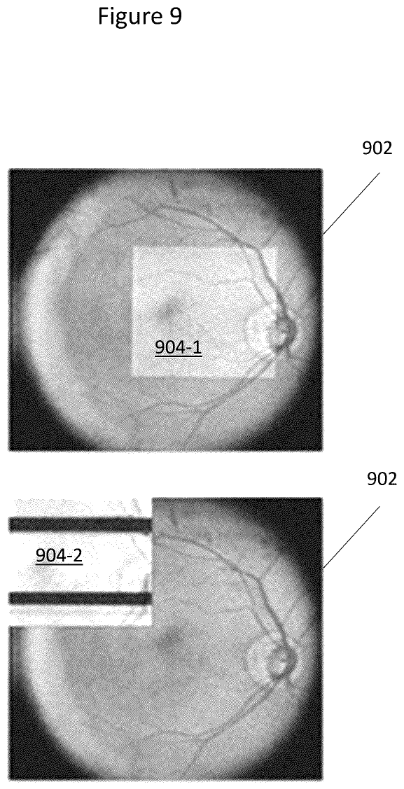

For example, with reference to FIG. 9, real-time enface image 904-2 has multiple black bands, indicating a patient's blink, whereas real-time enface image 904-1 does not have any black bands and is registered to a reference fundus image 902. The processor 204 can also implement a blinking/instability/movement threshold test to determine when it is proper to acquire real-time images or an OCT image. For example, the processor 204 can detect the patient not blinking/being stable and/or in the correct position for a predetermined time before acquiring real-time images or OCT images. For instance, if the patient has not blinked for a number of seconds and is correctly positioned in the view port 211.

In some embodiments, an LED display, or the like, viewable from view port 211 can generate a focal point for the patient to focus on during the imaging process. The LED display can be overlayed on the view port 211 optics, allowing the patient to stay in the view port 211 during the imaging process. The LED display could also generate flashing or steady lights to indicate the different statuses of the process. For instance, a green light to indicate that the image is acceptable or a yellow light to indicate that the image is not yet acceptable.

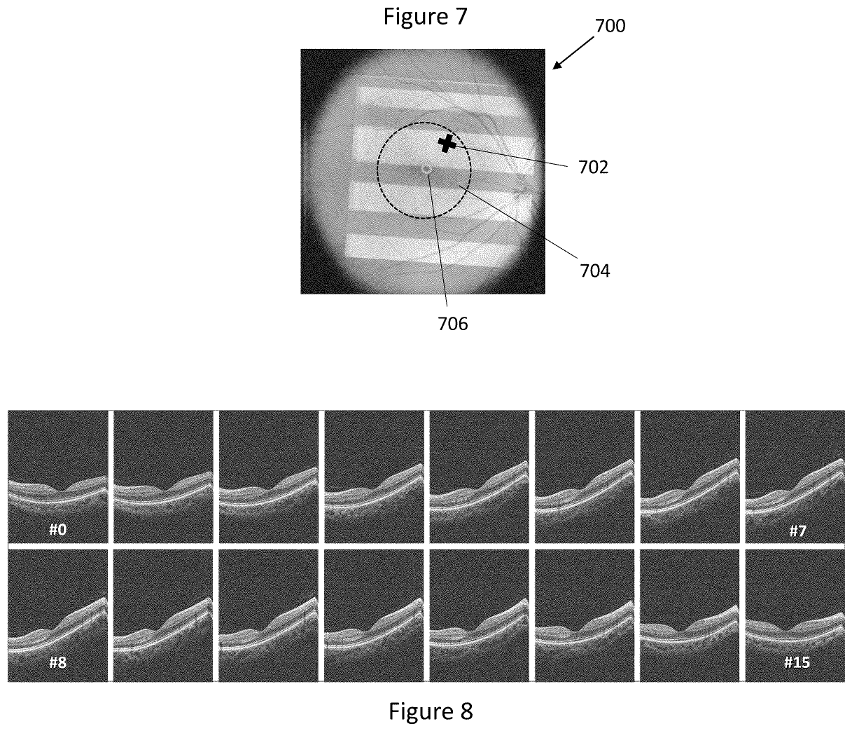

When an automated scan meets acceptability (quality) standards, the OCT imaging system 212 automatically acquires OCT images 305 at the determined desired scan location. For instance, finding the real-time image 700 acceptable, the automated imaging terminal 200, can then acquire OCT images by adjusting (e.g., centering) the view port 211 and/or OCT imaging system 212 on the same scan center 706 as the real-time image 700. For example, referencing FIG. 8, the OCT imaging system 212 can automatically acquire 16 structural OCT B-scans, numbered #0-#15, centered on the scan center 706 of the real-time image 700 or the desired scan location 702. The automated imaging terminal 200 can use actuators, motors, and the like, to adjust the OCT imaging system accordingly. In another embodiment, the automated imaging terminal 200 keeps the OCT imaging system 212 and the real-time imaging system 210 centered at the same position throughout the process. Therefore, when the automated real-time scans meet the desired threshold, the automated imaging terminal 200 is in position to acquire OCT images.

The automated imaging terminal 200 can acquire OCT images 305 using various scan patterns and techniques, for example, radial scans, circle scans, vertical scans, horizontal scans, or the like. These scan patterns may be indicated in the scan settings acquired with the reference image, and may also be unique or specialized to each patient. Thus, if a particularly scanning method/pattern is desired by the clinician or is better suited for that particular patient or pathology, that scanning method/pattern may be automatically indicated to and utilized by the automated imaging terminal 200.

The computer 202 can also store the acquired OCT images locally to the storage 206a or to the remote database 206b. In some embodiments, the computer 202 can store the acquired OCT images in local storage 206a for upload to the remote database 206b at a later time. For instance, if the internet connection was dropped or disrupted during the scan, the automated imaging terminal 200 can finish the scanning process, save the acquired OCT images locally, and upload the OCT images to the remote database at a later time when internet connectivity is restored. The computer 202 can link the acquired OCT images to the patient using the previously acquired patient information. In some embodiments, the OCT images are saved to the patient's file on the remote database 206b, to be later reviewed by a clinician. The clinician can then modify the desired scan location in the reference image or the instructions for the automated imaging terminal 200 if the clinician wishes.

This allows for continuous review and feedback from the clinician without having to schedule an appointment. The clinician could also include instructions to be displayed to the patient on the next visit to the automated imaging terminal 200. For instance, if the clinician sees an abnormality, the clinician could present a reminder or notification to the user to contact the clinician for a further evaluation. The reminder or notification can be displayed on the device via the view port 211 or on an information LED display located on the automated imaging terminal 200. In some embodiments, the reminder or notification can be sent directly to the patient using other forms of communication, for example, the patient's mobile device.

Using the example method described above, the automated imaging terminal 200 acquires OCT images 305 that are unique to each patient because the method can depend on information specific to that patient. For example, the patient's reference image can be marked by their clinician who can perform an exam on the patient and know specifics about that patient's pathology. This information may be stored with and/or associated with the patient's unique reference image, so that it may be acquired and used by the automated imaging terminal 200.

While various features are present above, it should be understood that the features may be used singly or in any combination thereof. Further, it should be understood that variations and modifications may occur to those skilled in the art to which the claimed examples pertain.

What is claimed is:

1. A method comprising:
receiving an input from a patient, and upon receiving the input:
acquiring a pre-existing reference image of an object from a remote database, the pre-existing reference image indicating a desired scan location;
acquiring personal information and/or scan settings regarding the patient from the remote database, the pre-existing reference image being unique to the patient and associated with personal information and/or scan settings;
acquiring a real-time image of the object;
registering the real-time image to the pre-existing reference image;
determining the desired scan location on the real-time image based on the registration; and
automatically acquiring an OCT image of the object at the desired scan location and according to the acquired personal information and/or scan settings.

2. The method of claim 1, wherein the pre-existing reference image was originally obtained by a clinician.

3. The method of claim 1, wherein the real-time image is an OCT en-face image.

4. The method of claim 1, further comprising:
authorizing the patient based on the input from the patient and acquired personal information.

5. The method of claim 1, wherein registering the real-time image and determining the desired scan location on the real time image is performed by a machine learning system.

6. The method of claim 1, wherein the OCT image at the desired scan location is automatically acquired based on whether the desired scan location is within a threshold range of a center of the real-time image.

7. The method of claim 6, further comprising:
determining the desired scan location is not within the threshold range of the center of the real-time image;
acquiring a second real-time image of the object;
registering the second real-time image to the reference image; and
determining the desired scan location on the second real-time image based on the registration of the second real-time image.

8. The method of claim 1, wherein the scan settings comprise a patient-specific scan pattern and the OCT image is automatically acquired according to the scan pattern.

9. The method of claim 1, further comprising:
aligning the OCT imaging system according to the desired scan location.

10. The method of claim 1, wherein the object is an eye.

11. A system comprising:
an optical coherence tomography (OCT) imaging system;
one or more processors collectively configured to:
receive an input from a patient, and upon receiving the input:
acquire a pre-existing reference image of an object from a remote databased in response to an input from a patient, the pre-existing reference image indicating a desired scan location;
acquire personal information and/or scan settings regarding the patient from the remote database, the pre-existing reference image being unique to the patient and associated with personal information and/or patient-specific scan settings;
acquire a real-time image of the object;
register the real-time image to the reference image;
determine the desired scan location on the real-time image based on the registration; and
automatically acquire an OCT image of the object at the desired scan location with the OCT imaging system according to the acquired personal information and/or scan settings.

12. The system of claim 11, wherein the reference image was originally obtained by a clinician.

13. The system of claim 11, wherein the real-time image is an OCT en-face image acquired with the OCT imaging system.

14. The system of claim 11, wherein the one or more processors are further collectively configured to:
authorize the patient's use of the system based on the input from the patient and the acquired personal information.

15. The system of claim 11, wherein the real-time image is registered to the reference image by one or more processors configured as a machine learning system.

16. The system of claim 11, wherein the OCT image at the desired scan location is automatically acquired based on whether the desired scan location is within a threshold range of a center of the real-time image.

17. The system of claim 16, wherein the one or more processors are further collectively configured to:
determine the desired scan location is not within the threshold range of the center of the real-time image;
acquire a second real-time image of the object;
register the second real-time image to the reference image; and
determine the desired scan location on the second real-time image based on the registration of the second real-time image.

18. The system of claim 11, wherein the scan settings comprise a patient-specific scan pattern, and the OCT image is automatically acquired according to the scan pattern.

19. The system of claim 11, wherein the one or more processors are further collectively configured to:
align the OCT imaging system according to the desired scan location.

20. The system of claim 11, wherein the object is an eye.
